(19) United States
(12) Patent Application Publication
(10) Pub. No.: US 2010/0234803 A1
(43) Pub. Date: Sep. 16, 2010

(54) ONE-TIME-USE AUTOMATIC SAFETY INJECTOR

(76) Inventors: Keun-Sik Kim, Seongnam-Si Gyeonggi-Do (KR); Tae-Sung Park, Nonsan-Si Chungnam (KR)

Correspondence Address: HYUN JONG PARK;Park & Associates IP Law LLC, 265 Bic Drive, Suite 106, Milford, CT, 06461, US

(21) Appl. No.: 12/300,317

(22) PCT Filed: Apr. 27, 2007
(86) PCT No.: PCT/KR07/02082

§ 371 Date: Nov. 10, 2008

(30) Foreign Application Priority Data
May 10, 2006 (KR) 10-2006-0041916

(51) Int. Cl. A61M 5/50

(52) U.S. Cl. 604/110

(57) ABSTRACT

Disclosed is a one-time use safety injector capable of easily removing a remaining solution in the administration of an injection solution and easily being detached and discarded after use. The automatic safety injector according to the present invention includes a needle flange 31 having a needle 30 mounted in the front thereof and a cone inner space 32 and an edge portion 33 formed in the rear thereof, a coupling ring 50 being inserted into a circumferential edge of the edge portion 33; an adapter 60 including a hanging projection 61, a cone protrusion 62, coupling ring insertion grooves 63, a spherical flange 64 and a gasket 70, the gasket 70 being inserted from the front circumferential edge up to the hanging projection 61, the cone protrusion 62 being projected and installed in its head portion, the coupling ring insertion grooves 63 being formed around the cone protrusion 62, and the spherical flange 64 being formed in the front of the adapter 60; a piston 40 including elastic wings 41,41′ and a spherical flange 42, the elastic wings 41,41′ being installed in both sides of the head portion of the piston 40, and the spherical flange 42 being projected and installed inside the piston 40; and an injector cylinder 20 having an injection solution inlet 21 formed in the front thereof and an adapter hanging projection 22 formed in the rear thereof.

### TECHNICAL FIELD

The present invention relates to a one-time use automatic safety injector capable of administering an injection solution without any of the remaining injection solution and easily being detached and discarded after use, and more particularly to a one-time use automatic safety injector capable of administering the whole quantity of an injection solution in an injector cylinder by forming an inner injection administration route of the needle flange equipped with a needle and a head portion of an adapter in a cone shape to closely adhere to each other in the administration of the injection solution, the needle being inserted and coupled with a front inlet of the injector cylinder; and of automatically separating a piston from the adapter after use by coupling a rear flange of the adapter with an elastic wings formed in a head portion of the piston to move forward/backward inside the injector cylinder, wherein the needle may be pulled into the injector cylinder by allowing the needle to move backward along with the adapter since the head portion of the adapter and the circumferential edge of the needle flange may be inserted and coupled with each other by means of a coupling ring.

### BACKGROUND ART

A conventional disposable injector is configured so that it can have an inner space portion; that an injection solution in the injector cylinder can be sucked, compressed and administered using an injector cylinder and a piston which is present in the injector cylinder and has a gasket coupled with a head portion thereof, the injector cylinder having an injection solution inlet formed with a stepped height in the front of the injector cylinder; that a needle flange equipped with a needle can be detachably coupled to a front injection solution inlet of the injector cylinder; and that a needle protection cap is detachably coupled to the needle to protect the needle.

The conventional disposable injector has no problem in its use in removing a protection cap and sucking, compressing and administering an injection solution. However, the conventional disposable injector has problems regarding some accidents. For example, users (medical practitioners) may get pricked by a needle in the procedure of separating the needle from the protection cap and sealing and discarding the needle after use, and workers may also frequently get pricked by a needle since the needle is uncovered from the protection cap when the disposable injector is discarded by special waste disposal companies. In addition, it has been reported in the medical profession that, since carriers of infectious pathogens in patient's blood are present in the used needle, the patient's carriers are transferred to the users and workers if the users and workers get pricked by the needle, and then the users and workers may be infected with the patient's infectious diseases (hepatitis, AIDS, etc.).

In order to solve the above problems regarding the common disposable injector, the conventional disposable injectors have been modified. Here, the modified disposable injectors are configured so that a needle flange equipped with a needle can be inserted into an injector cylinder and pushed toward an injection solution inlet of the injector cylinder to couple the needle flange with the injection solution inlet, and a piston equipped with an adapter can be used to suck, compress and administer an injection solution. Also, the modified disposable injectors are configured so that the hanging protrusion or the hanging projection can be projected and formed in a head portion of the adapter and a cutting groove or a plurality of holes can be punched in a front portion of the piston coupled with the adapter. Therefore, if a piston is coupled with a prominence formed inside the needle flange by means of the hanging protrusion (the hanging projection) and moves backward after use of the disposable injectors, then the disposable injectors may be discarded by pulling the needle flange equipped with a needle into the injector cylinder, pulling back the piston outward of the injector cylinder and cutting the cutting groove (a plurality of the holes) of the piston, which is pulled outward, thereby to detach the piston from the injector cylinder.

The modified disposable injector has an advantage that accidents of getting pricked by a needle may be prevented since the needle flange equipped with a needle is pulled into the injector cylinder by means of a hanging protrusion after use, the hanging protrusion being projected toward a head portion of the adapter. However, the disposable injector has a problem that it has a high defect rate in its production and it is cut or partially damaged because the hanging protrusion of the adapter coupled with the needle flange is formed projectedly. Also, the disposable injector has a problem that it is impossible to administer a suitable amount of the injection solution in addition to wasting an expensive injection solution because the injection solution, which was sucked into the injector cylinder, is not completely administered due to the presence of the formed space between an inner space of the needle flange and the head portion of the adapter, and that the discarding procedure is rather troublesome because a user may apply pressure to a region where a cutting groove (a plurality of holes) is formed to cut out a piston.

### DISCLOSURE OF INVENTION

### Technical Problem

Accordingly, the present invention is designed to solve the problems of the prior art, and therefore it is an object of the present invention to provide a one-time use safety injector capable of administering an injection solution without any of the remaining injection solution and simultaneously administering a suitable amount of the injection solution by coupling a head portion of an adapter with an inner space of a needle flange in a cone shape; of being used and easily discarded by being automatically disassembled as a piston moves backward because an adapter and a piston are separately manufactured and assembled into a safety injector; of confirming administration of the injection solution by sensing the administration of the injection solution by user's hands and hearing a faint alarm generated in the administration of the injection solution because the needle flange and the head portion of the adapter are coupled with each other by means of the coupling ring; and of pulling a needle flange and an adapter in the inside of an injector cylinder, the needle flange being equipped with a needle coupled by means of the coupling ring.

### Technical Solution

In order to accomplish the above object, the present invention provides a one-time use automatic safety injector (N) including a needle protection cap 10 and an injector cylinder 20, wherein the one-time use automatic safety injector (N) is discarded after a needle flange 31 equipped with a needle 30 is pulled into the injector cylinder 20 and a piston 40 is detached from the injector cylinder 20, the one-time use automatic safety injector (N) including:

a needle flange 31 having a needle 30 mounted in the front thereof and a cone inner space 32 and an edge portion 33 formed in the rear thereof, a coupling ring 50 being inserted into a circumferential edge of the edge portion 33;

an adapter 60 including a hanging projection 61, a cone protrusion 62, coupling ring insertion grooves 63, a spherical flange 64 and a gasket 70, the gasket 70 being inserted from the front circumferential edge up to the hanging projection 61, the cone protrusion 62 being projected and installed in its head portion, the coupling ring insertion grooves 63 being formed around the cone protrusion 62, and the spherical flange 64 being formed in the front of the adapter 60;

a piston 40 including elastic wings 41,41′ and a spherical flange 42, the elastic wings 41,41′ being installed in both sides of the head portion of the piston 40, and the spherical flange 42 being projected and installed inside the piston 40; and

an injector cylinder 20 having an injection solution inlet 21 formed in the front thereof and an adapter hanging projection 22 formed in the rear thereof.

Also, it is characterized in that the edge portion 33 of the needle flange 31 and the coupling ring insertion grooves 63 of the adapter 60 are formed aslope to easily couple with the coupling ring 50 having one open side and a twisted shape.

And, it is characterized in that the hanging projection 22 formed in the rear of the injector cylinder 20 has an internal portion formed at a gentle slope and an external portion formed at a steep slope.

In addition, it is characterized in that the coupling ring 50 is formed in a washer type with one open side and made of stainless steel and synthetic resin.

### ADVANTAGEOUS EFFECTS

As described above, the one-time use automatic safety injector according to the present invention can be useful to solve the problem that a cutting groove of the conventional piston and a region of punched holes are cut with a pressure by separately manufacturing an adapter and a piston to be easily assembled and detached automatically; to prevent a waste of expensive injection solution and simultaneously administer a suitable amount of the injection solution by forming a head portion of the adapter and an inner space of the needle flange in a cone shape; to safely pull a needle flange equipped with a needle into an inner space of the injector cylinder in the coupling process using a coupling ring; and to confirm the administration of the injection solution with a tactile sense by user's hands and an auditory sense; etc.

### BRIEF DESCRIPTION OF THE DRAWINGS

These and other features, aspects, and advantages of preferred embodiments of the present invention will be more fully described in the following detailed description, taken accompanying drawings. In the drawings:

FIG. 1 is an exploded view illustrating an assembly of a one-time use automatic safety injector according to the present invention;

FIG. 2 is a cross-sectional view showing a coupling configuration of a needle flange and an adapter according to the present invention;

FIG. 3 is a configurational view showing that a piston is detached according to the backward motion of the one-time use automatic safety injector according to the present invention;

FIG. 4 is a partial cross-sectional view showing that the piston moves backward in the one-time use automatic safety injector according to the present invention; and

FIG. 5 is a use diagram showing an operation order of the one-time use automatic safety injector according to the present invention.

### BEST MODE FOR CARRYING OUT THE INVENTION

Hereinafter, preferred embodiments of the present invention will be described in detail referring to the accompanying drawings.

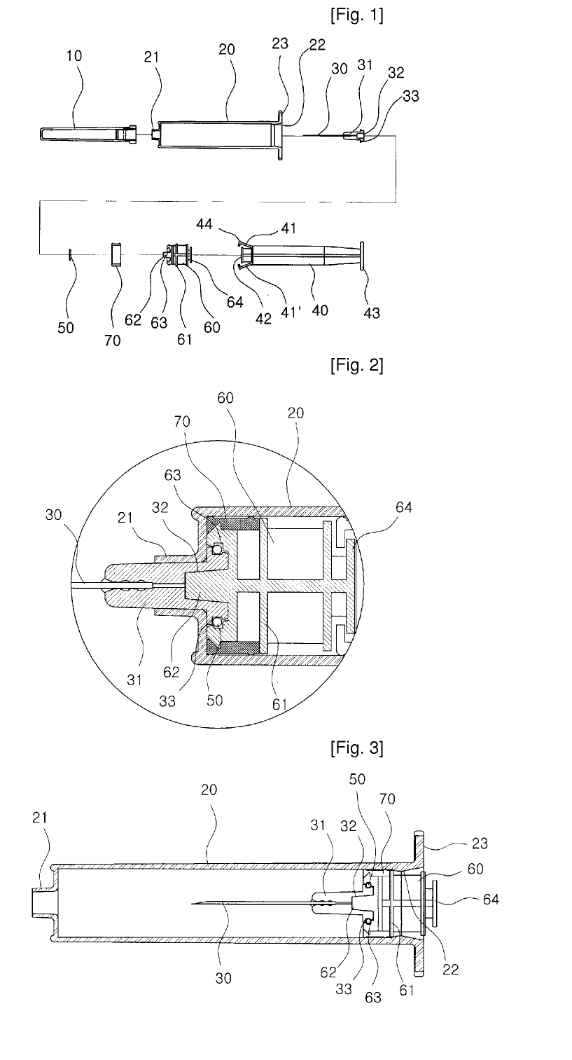

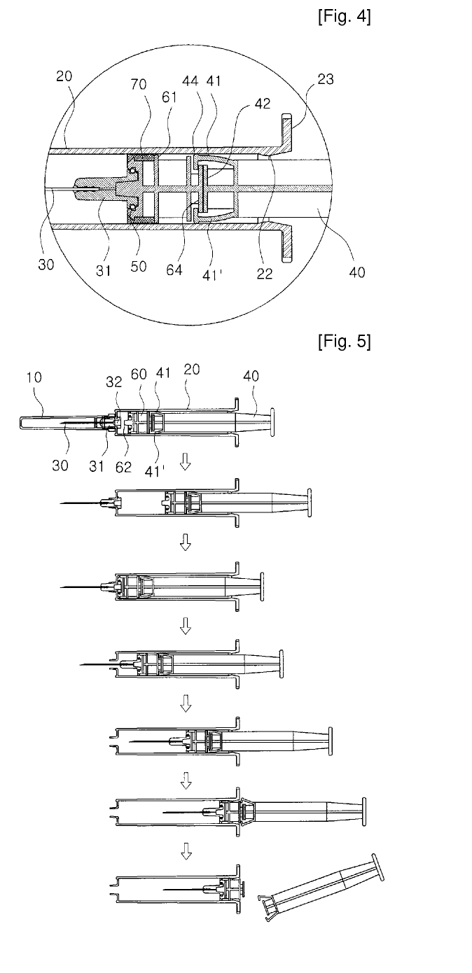

FIG. 1 is an exploded view showing an assembly of a one-time use automatic safety injector according to the present invention, and shows that the one-time use automatic safety injector includes a protection cap, an injector cylinder, a needle flange equipped with a needle, a coupling ring, a gasket, adapter and a piston. FIG. 2 is a cross-sectional view showing a coupling configuration of a needle flange and an adapter according to the present invention, and shows that a head portion of the adapter is coupled with an injection solution inlet of the injector cylinder by means of a coupling ring which is inserted into a circumferential edge of the needle flange equipped with a needle. FIG. 3 is a configurational view showing that a piston is detached according to the backward motion of the one-time use automatic safety injector according to the present invention, and shows that the piston coupled with the rear of the adapter is automatically disassembled after use of the one-time use injector as the needle flange equipped with a needle moves backward along with the adapter in order to pull the needle into the injector cylinder since the adapter is coupled with the needle flange by means of the coupling ring. FIG. 4 is a partial cross-sectional view showing that the piston moves backward in the one-time use automatic safety injector according to the present invention, and shows that the piston moves backward along with the needle flange since the piston is coupled with the needle flange by means of the coupling ring in the previous step of FIG. 3. FIG. 5 is a use diagram showing an operation order of the one-time use automatic safety injector according to the present invention, and shows steps of sucking an injection solution; administering the injection solution; performing sequential backward motions of a piston; and disassembling the piston in the one-time use injector manufactured according to the present invention.

The one-time use automatic safety injector (N) includes a needle protection cap 10 and an injector cylinder 20, wherein the one-time use automatic safety injector (N) is discarded after a needle flange 31 equipped with a needle 30 is pulled into the injector cylinder 20 and a piston 40 is detached from the injector cylinder 20, the one-time use automatic safety injector (N) including:

a needle flange 31 having a needle 30 mounted in the front thereof and a cone inner space 32 and an edge portion 33 formed in the rear thereof, a coupling ring 50 being inserted into a circumferential edge of the edge portion 33;

an adapter 60 including a hanging projection 61, a cone protrusion 62, coupling ring insertion grooves 63, a spherical flange 64 and a gasket 70, the gasket 70 being inserted from the front circumferential edge up to the hanging projection 61, the cone protrusion 62 being projected and installed in its head portion, the coupling ring insertion grooves 63 being formed around the cone protrusion 62, and the spherical flange 64 being formed in the front of the adapter 60;

a piston 40 including elastic wings 41,41′ and a spherical flange 42, the elastic wings 41,41′ being installed in both sides of the head portion of the piston 40, and the spherical flange 42 being projected and installed inside the piston 40; and

an injector cylinder 20 having an injection solution inlet 21 formed in the front thereof and an adapter hanging projection 22 formed in the rear thereof.

The edge portion 33 of the needle flange 31 and the coupling ring insertion grooves 63 of the adapter 60 were formed aslope to easily couple with the coupling ring 50 having one open side and a twisted shape.

The hanging projection 22 formed in the rear of the injector cylinder 20 had an internal portion formed at a gentle slope and an external portion formed at a steep slope.

The coupling ring 50 had a washer type with one open side and was made of stainless steel and synthetic resin.

Reference numerals 23, 43 and 44, which are not described in the accompanying drawings, represent an injector cylinder flange, a piston flange and a protrusion of an elastic wing, respectively.

As described above, the one-time use automatic safety injector according to the present invention can be useful to confirm the administration of the injection solution using tactile and auditory senses of users in administering the injection solution, to solve the problem of wasting the expensive injection solution and administer a suitable amount of the injection solution since the injection solution in the injector cylinder is administered completely, to prevent accidents of being infected when a user gets pricked by a needle, and to automatically disassemble a piston by pulling the needle in the inside of the injector cylinder in the disposal step after use.

As shown in FIG. 1, the one-time use safety injector is composed of a needle protection cap 10, an injector cylinder 20, a needle flange 31 equipped with a needle 30, a coupling ring 50, a gasket 70, an adapter 60 and a piston 40.

The needle protection cap 10 is universal and serves to prevent accidents that users get pricked by a needle 30 by protecting the needle 30 that is projectedly installed in the injection solution inlet 21 of the injector cylinder 20.

The injector cylinder 20 has an injection solution inlet 21 formed in the front thereof; a hanging projection 22 for an adapter formed in the rear thereof; and an injector cylinder flange 23 formed in the rear of the hanging projection 22. And, the adapter hanging projection 22 has an internal portion formed at a gentle slope and an external portion formed at a steep slope, and therefore it is configured so that an adapter 60, as described later, is smoothly pulled in when the adapter 60 is pulled into the injector cylinder 20 but not pulled out of the injector cylinder 20 since the hanging projection 61 of the adapter 60 is hanged over the hanging projection 22 of the injector cylinder 20.

The needle flange 31 equipped with a needle is inserted up to the injection solution inlet 21 of the injector cylinder 20 through the inside of the injector cylinder 20, and the piston 40 is fit and inserted into the rear of the injector cylinder 20, wherein the piston 40 has elastic wings 41,41′ and the adapter 60 which the gasket 70 is coupled. (see the first drawing of FIG. 5)

The adapter 60 serves to insert the coupling ring 50 into the coupling ring insertion groove 63 since a cone protrusion 62 is formed in a head portion the adapter 60 and a coupling ring insertion groove 63 is formed outside the adapter 60.

At this time, the coupling ring 50 has one open side and is manufactured in a washer type having a twisted shape, and the coupling ring insertion groove 63 of the adapter 60 may serve to safely install the coupling ring 60 since the front of the coupling ring insertion groove 63 is formed aslope.

Because the spherical flange 64 formed in the rear of the adapter 60 brings face to face with the spherical flange 42 of the piston 40, the spherical flange 64 of the adapter 60 is clamped due to elasticity of the elastic wings 41,41′ formed in both outer sides if the spherical flange 64 formed in the rear of the adapter 60 is pulled inward the injector cylinder 20.

In the case of the injector (N) assembled as described above, an injection solution is sucked into the injector cylinder 20 if the piston 40 moves backward as shown in the second drawing of FIG. 5, and the injection solution is injected into a patient if the piston 40 moves forward as shown in the third drawing of FIG. 5.

At this time, the whole injection solution sucked in the injector cylinder 20 may be administered by inserting the spherical protrusion 62, formed in a head portion of the adapter 60, into an inner space of the needle flange 31, that is, the cone inner space 32. At the same time, the injector (N) user's hands are sensed and a faint alarm is generated when the coupling ring 50 installed in the coupling ring insertion groove 63 of the adapter 60 is inserted into the edge portion 33 of the needle flange 31. Therefore, the piston 40 should not be pushed to move forward any more since the injector (N) user recognizes that the injection solution in the injector (N) is completely administered.

As in the above-mentioned operation of the injector (N), because the edge portion 33 of the needle flange 31 is formed aslope, the coupling ring 50 is easily coupled with the needle flange 31 when the coupling ring 50 installed in the coupling ring insertion groove 63 of the adapter 60 is inserted into the edge portion 33 of the needle flange 31, and the coupling ring 50 may move backward with its being engaged with the needle flange 31 without disassembly when the piston 40 moves backward.

When the administration of the injection solution is completed, the needle 30 is pulled out of a patient, and the piston 40 moves backward, as shown in the third to sixth drawings of FIG. 5.

Referring to the backward motion, the adapter 60 and the needle flange 31 are pulled into the injector cylinder 20 since they are firmly coupled with each other by means of the coupling ring 50, whereas the needle flange 31 equipped with a needle 30 continues to move backward while it is drooped downwardly, as shown in FIG. 3. Then, the hanging projection 61 of the adapter 60 moves to the end of the injector cylinder 20 while it is hang over the hanging projection 22 of the injector cylinder 20, and the elastic wings 41,41′ of the piston 40 are pulled out of the injector cylinder 20, as shown in FIG. 4. At the same time, the piston 40 is disassembled from the needle flange 31 by means of an elastic force of the elastic wings 41,41′, as shown in the seventh drawing of FIG. 5.

The needle flange 31 equipped with a needle 30 may not be basically re-used since it is pulled in a drooped state by means of the coupling ring 50 having a twisted shape while being pulled into the injector cylinder 20.

The protrusion 44 formed in the elastic wing 41,41′ serves to reduce a friction force by point-contacting a piston with the inner wall of the injector cylinder 20 when the piston 40 moves forward or backward, and therefore the piston 40 smoothly moves forward or backward.

The present invention has been described in detail. However, it should be understood that the detailed description and specific examples, while indicating preferred embodiments of the invention, are given by way of illustration only, since various changes and modifications within the spirit and scope of the invention will become apparent to those skilled in the art from this detailed description.

1. A one-time use automatic safety injector (N) including a needle protection cap 10 and an injector cylinder 20, wherein the one-time use automatic safety injector (N) is discarded after a needle flange 31 equipped with a needle 30 is pulled into the injector cylinder 20 and a piston 40 is detached from the injector cylinder 20, the one-time use automatic safety injector (N) comprising:
a needle flange 31 having a needle 30 mounted in the front thereof and a cone inner space 32 and an edge portion 33 formed in the rear thereof, a coupling ring 50 being inserted into a circumferential edge of the edge portion 33;
an adapter 60 including a hanging projection 61, a cone protrusion 62, coupling ring insertion grooves 63, a spherical flange 64 and a gasket 70, the gasket 70 being inserted from the front circumferential edge up to the hanging projection 61, the cone protrusion 62 being projected and installed in its head portion, the coupling ring insertion grooves 63 being formed around the cone protrusion 62, and the spherical flange 64 being formed in the front of the adapter 60;
a piston 40 including elastic wings 41,41′ and a spherical flange 42, the elastic wings 41,41′ being installed in both sides of the head portion of the piston 40, and the spherical flange 42 being projected and installed inside the piston 40; and
an injector cylinder 20 having an injection solution inlet 21 formed in the front thereof and an adapter hanging projection 22 formed in the rear thereof.

2. The one-time use automatic safety injector according to claim 1, wherein the edge portion 33 of the needle flange 31 and the coupling ring insertion grooves 63 of the adapter 60 are formed aslope to easily couple with the coupling ring 50 having one open side and a twisted shape.

3. The one-time use automatic safety injector according to claim 1, wherein the hanging projection 22 formed in the rear of the injector cylinder 20 has an internal portion formed at a gentle slope and an external portion formed at a steep slope.

4. The one-time use automatic safety injector according to claim 1, wherein the coupling ring 50 is formed in a washer type with one open side and made of stainless steel and synthetic resin.
